(19) United States
(12) Patent Application Publication
(10) Pub. No.: US 2026/0083389 A1
(43) Pub. Date: Mar. 26, 2026

(54) SYSTEMS AND METHODS FOR EMBEDDING GAMMA WAVE FREQUENCIES INTO MUSICAL COMPOSITIONS

(71) Applicant: Esperer, Inc., Newton Centre, MA (US)

(72) Inventors: Westley Barry Nolin, Newton Centre, MA (US); Thomas Provencher, Newton Centre, MA (US)

(73) Assignee: Esperer, Inc., Newton Centre, MA (US)

(21) Appl. No.: 19/331,044

(22) Filed: Sep. 17, 2025

Related U.S. Application Data
(60) Provisional application No. 63/697,337, filed on Sep. 20, 2024.

(51) Int. Cl. A61B 5/38; A61B 5/374; G06F 3/01

(52) U.S. Cl. CPC A61B 5/38; A61B 5/374; G06F 3/015

(57) ABSTRACT

The present application is directed to a system and method for generating a therapeutic musical composition. The system includes a signal generator configured to generate a gamma wave frequency signal, a memory configured to store computer-executable instructions, and a data processing system. The data processing system executes instructions to perform a method that includes altering the gamma wave frequency signal to tailor desired musical parameters, integrating the altered gamma wave frequency signal into a musical composition to create a gamma wave signal-integrated musical composition, and mastering the gamma wave signal-integrated musical composition to enhance an aspect of the gamma wave signal-integrated musical composition.

### CROSS-REFERENCE TO RELATED APPLICATIONS

This application claims the benefit under 35 U.S.C. 119(e) of U.S. Provisional Ser. No. 63/697,337 filed Sep. 20, 2024, the entire disclosure of which is hereby incorporated herein by reference.

### BACKGROUND

The following description is provided to assist the understanding of the reader. None of the information provided or references cited is admitted to be prior art.

The interplay between auditory stimuli and neurological responses has long fascinated researchers, with various studies exploring how different sound frequencies can influence brain activity and cognitive functions. Among these, gamma waves, which oscillate, for example, between 30 to 100 Hz, are associated with higher cognitive processes such as attention, memory, and consciousness. Recent advancements in neuro-acoustic research suggest that embedding gamma waves into music enhances these cognitive functions by leveraging the brain's natural responsiveness to these frequencies. This application addresses a novel approach to integrating gamma wave frequencies into musical compositions, offering a method to potentially enhance mental performance and emotional well-being through structured auditory experiences.

### SUMMARY

The techniques disclosed herein provide methods and systems for generating musically pleasing audio compositions having embedded gamma wave frequencies that provide therapeutic benefits. One aspect of the present disclosure describes a method for generating a therapeutic musical composition. The method includes generating a gamma wave frequency signal, altering the gamma wave frequency signal to tailor desired musical parameters, integrating the altered gamma wave frequency signal into a musical composition to create a gamma wave signal-integrated musical composition, and mastering the gamma wave signal-integrated musical composition to enhance an aspect of the gamma wave signal-integrated musical composition.

In another aspect, the method further includes administering the mastered gamma wave-signal integrated musical composition to an individual via a speaker. Integrating the altered gamma wave frequency signal into a musical composition according to such a method may further includes at least one of modulating an amplitude or a frequency of the altered gamma wave frequency signal. Additionally or alternatively, integrating the altered gamma wave frequency signal into a musical composition may include layering the altered gamma wave frequency signal into the musical composition as a background audio track.

Yet another aspect of the present disclosure describes a system that includes a signal generator configured to generate a gamma wave frequency signal, a memory configured to store computer-executable instructions, and a data processing system coupled to the signal generator and the memory, wherein the data processing system is configured to execute the computer-executable instructions to perform various operations. These operations may include altering the gamma wave frequency signal to tailor desired musical parameters, integrating the altered gamma wave frequency signal into a musical composition to create a gamma wave signal-integrated musical composition, and mastering the gamma wave signal-integrated musical composition to enhance an aspect of the gamma wave signal-integrated musical composition.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 depicts a flow chart of an example process for creating a gamma-wave embedded musical composition in accordance with an illustrative embodiment.

FIG. 2 depicts a block diagram of an example system for creating a gamma-wave embedded musical composition using the process of FIG. 1, in accordance with an illustrative embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

This application describes various techniques for embedding gamma wave frequencies into musical compositions. One example technique involves generating or modulating gamma wave signals and embedding these signals into a musical framework to create an auditory experience that is both musically pleasing and neurologically stimulating. The approach includes the steps of generating gamma wave frequencies, integrating these frequencies into music tracks, and producing the final musical output. The gamma wave frequencies can be embedded in various forms, including but not limited to, amplitude modulation, frequency modulation, or as a background audio layer, ensuring that the gamma waves are incorporated without disrupting the musical harmony or integrity of the original song.

Reference is now made to the figures. The figures depict various systems and methods. In alternative embodiments, one or more components or steps in the figures may be omitted or moved. It should be understood that like reference numerals can refer to like elements throughout, repetitive descriptions of which can be omitted. It should be also noted that in the drawings, the dimensions of the features are not intended to be to true scale and can be exaggerated for the sake of allowing greater understanding.

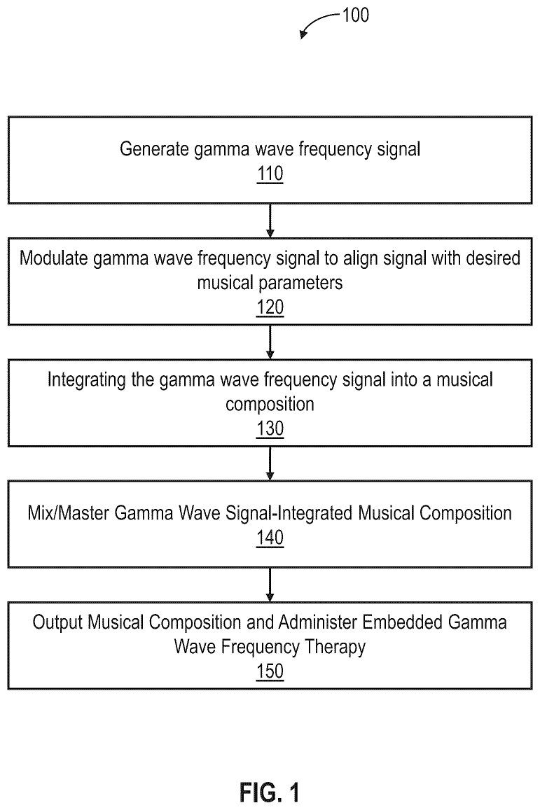

To achieve effective embedding of gamma waves into music, the invention uses a multi-step process. FIG. 1 depicts a flow chart of an example process 100 for creating a gamma-wave embedded musical composition in accordance with an illustrative embodiment. In an operation 110, a signal comprising gamma wave frequencies is generated using a suitable signal generator. In an embodiment, the signal generator may include one or more oscillatory wave generators or synthesizers and produces signals with a frequency within the 30 to 100 Hz range.

In an operation 120, this signal (including its component gamma waves) is modulated and/or otherwise adjusted by a signal modulator to align the signal with specific, desired musical parameters such as tempo, key, and rhythm to maintain harmony and enhance an overall auditory experience of a listener. This operation may include performing a warping operation that allows for the signal and associated audio to be selectively time-stretched. In an embodiment, such operations may be accomplished using a software program with one or more warp modes that provide specific tailored ways of selecting, overlapping, and crossfading to alter how an audio signal is stretched and/or compressed.

In an operation 130, the signal (including its component gamma waves) is integrated into a musical composition utilizing any suitable manner of integration. One example approach for integrating the gamma wave signal into the musical composition may include amplitude modulation, whereby the amplitude of the gamma wave signal is used to modulate a volume of musical elements within the musical composition, thereby creating a desirable interplay between the musical composition and the therapeutic gamma wave signal. In another example approach, frequency modulation techniques may be employed to integrate the gamma wave signal into the musical composition. According to such an approach, gamma wave frequencies are used to modulate specific aspects of the musical frequencies of the musical composition, thereby creating a synergistic effect from the combination of the gamma wave frequencies and the musical composition that enhances the listener's cognitive engagement while also providing the therapeutic benefits associated with gamma wave frequencies.

Still another example manner of integrating the gamma wave signal into the musical composition involves layering the gamma wave frequency signal into the musical composition as a background audio track that complements the primary musical elements of the composition. In an embodiment, sound engineering techniques are utilize to ensure layering of the background audio track without overpowering or distorting the primary musical elements of the composition. Layering of the gamma wave frequency signal may including adding a white noise layer, adding a release layer, adding layers across multiple octaves, or other layering techniques to enhance musicality. Additional layering strategies may be further utilized in operation 130, including group compression techniques, group multiband compression techniques, group saturation techniques, etc.

In an operation 140, the gamma wave signal-integrated musical composition is mixed and mastered to ensure that the integration of gamma waves is both effective and unobtrusive, thereby resulting in an musically pleasing composition. This operation involves balancing the musical composition to achieve the desired therapeutic (e.g., cognitive and emotional) effects while preserving the musical integrity and listenability of the musical composition. The end-product is a composition of music that not only provides aesthetic enjoyment but also provides therapeutic benefits, including enhancing cognitive functions (e.g., attention, memory, problem-solving skills, etc.) and emotional well-being through the embedded gamma wave frequencies.

In an operation 150, the mixed and/or mastered gamma wave signal-integrated musical composition is output to an audio playing device so that the musical composition may be listened to by an individual and therapy associated with the embedded gamma wave frequencies thereby administered to the individual.

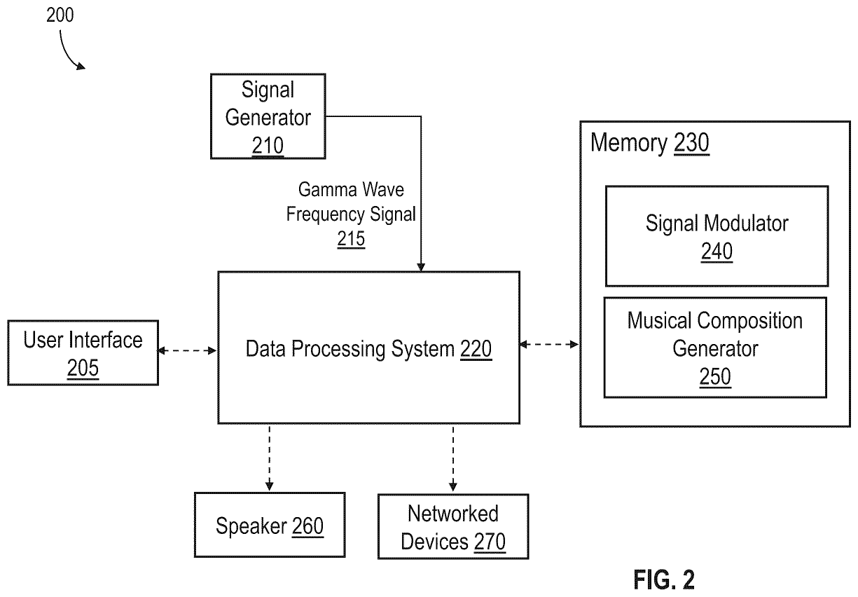

FIG. 2 depicts a block diagram of an example system 200 for creating a gamma-wave embedded musical composition using the process of FIG. 1, in accordance with an illustrative embodiment. The system 200 shown in FIG. 2 is simplified for illustrative purposes, and thus, can be implemented as any of various other configurations while remaining within the scope of the present disclosure. In some embodiments, the system 200 can include more, fewer, or different components than shown in FIG. 2.

The system 200 includes a signal generator 210 that is configured to generate one or more signals comprising gamma wave frequencies (i.e., gamma wave frequency signal 215). In an embodiment, signal generator 210 may include one or more oscillatory wave generators or synthesizers and produces signals having a frequency within the 30 to 100 Hz range. Signal generator 210 provides gamma wave frequency signal 215 to a data processing system 220.

Data processing system 220 may be any computing device including one or more processors coupled with memory and software and configured to perform the various processes and tasks described herein, including the operations described above with respect to FIG. 1. The data processing system 220 is in communication with the signal generator 210, a user interface 205, a memory 230, a speaker 260, and one or more networked devices 270. In some embodiments, the data processing system 220 can be in communication with the various other components of system 200 via any suitable connection, including via a physical connection, via a wired network, via a wireless network, via a cloud service, etc.

One or more processors of the data processing system 220, operating individually or collectively, runs software stored on coupled memory to perform various operations as described above with respect to FIG. 1. In alternative embodiments, fewer or additional operations may be performed by data processing system 220. For example, data processing system 220 may execute computer-readable software instructions stored in a signal modulator portion 240 of memory 230 to modulate or otherwise adjust the gamma wave frequency signal 215 received from signal generator 210 to align the signal with specific, desired musical parameters such as tempo, key, and rhythm to maintain harmony and enhance an overall auditory experience of a listener. The associated software instructions may include instructions for performing a warping operation that allows for the signal and associated audio to be selectively time-stretched and may further utilize one or more warp modes that provide specific tailored ways of selecting, overlapping, and crossfading to alter how an audio signal is stretched and/or compressed.

Data processing system 220 may further execute computer-readable software instructions stored in a musical composition generator portion 250 of memory 230 to integrate the modulated and/or adjusted gamma wave frequency signal 215 into a musical composition utilizing any suitable manner of integration. The musical composition may be stored in memory 230 or received from one or more networked device 270. As discussed for FIG. 1, one example approach for integrating the gamma wave frequency signal 215 into the musical composition may include an amplitude modulation technique or a frequency modulation technique. Additionally, data processing system 220 may execute software instructions that utilize a layer technique to add a gamma wave frequency signal 215 to the musical composition as a background audio track. Data processing system 220 may receive instructions from a user via a user interface 205 to control operations and specific parameters of the gamma wave frequency signal integration process. Using such an approach, gamma wave frequencies in the gamma wave frequency signal 215 modulate specific aspects of the musical frequencies of the musical composition, thereby creating a synergistic effect from the combination of the gamma wave frequencies and the musical composition that enhances the listener's cognitive engagement while also providing the therapeutic benefits associated with gamma wave frequencies.

The data processing system 220 further executes software instructions (optionally in combination with direction provided via user interface 205) to mix and/or master the gamma wave signal-integrated musical composition. In this way, the data processing system 220 provides a musically pleasing composition by ensuring that the integration of gamma waves is both effective and unobtrusive. To do so, the data processing system 220 may balance the musical composition to achieve the desired therapeutic (e.g., cognitive and emotional) effects while preserving the musical integrity and listenability of the musical composition. The end-product is a composition of music that not only provides aesthetic enjoyment but also provides therapeutic benefits, including enhancing cognitive functions (e.g., attention, memory, problem-solving skills, etc.) and emotional well-being through the embedded gamma wave frequencies.

The data processing system 220 is further communicatively coupled to the speaker 260 and/or one or more networked devices 270 to which the data processing system 220 may communicate the mixed and/or mastered gamma wave signal-integrated musical composition. Any of the speaker 260 and the networked devices 270 may include components to play the musical composition to an individual such that the individual may enjoy the music while therapy associated with the embedded gamma wave frequencies is thereby administered to the individual.

The foregoing description of illustrative embodiments has been presented for purposes of illustration and of description. It is not intended to be exhaustive or limiting with respect to the precise form disclosed, and modifications and variations are possible in light of the above teachings or can be acquired from practice of the disclosed embodiments.

While certain embodiments have been illustrated and described, it should be understood that changes and modifications can be made therein in accordance with ordinary skill in the art without departing from the technology in its broader aspects as defined in the following claims.

The embodiments, illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, compounds, compositions or systems, which can of course vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Additional embodiments can be set forth in the following claims.

What is claimed is:

1. A method for generating a therapeutic musical composition, the method comprising:
generating a gamma wave frequency signal;
altering the gamma wave frequency signal to tailor desired musical parameters;
integrating the altered gamma wave frequency signal into a musical composition to create a gamma wave signal-integrated musical composition; and
mastering the gamma wave signal-integrated musical composition to enhance an aspect of the gamma wave signal-integrated musical composition.

2. The method of claim 1, further comprising administering the mastered gamma wave-signal integrated musical composition to an individual via a speaker.

3. The method of claim 1, wherein integrating the altered gamma wave frequency signal into a musical composition comprises at least one of modulating an amplitude or a frequency of the altered gamma wave frequency signal.

4. The method of claim 1, wherein integrating the altered gamma wave frequency signal into a musical composition comprises layering the altered gamma wave frequency signal into the musical composition as a background audio track.

5. A system comprising:
a signal generator configured to generate a gamma wave frequency signal;
a memory configured to store computer-executable instructions;
a data processing system coupled to the signal generator and the memory, wherein the data processing system is configured to execute the computer-executable instructions to:
alter the gamma wave frequency signal to tailor desired musical parameters;
integrate the altered gamma wave frequency signal into a musical composition to create a gamma wave signal-integrated musical composition; and
master the gamma wave signal-integrated musical composition to enhance an aspect of the gamma wave signal-integrated musical composition.

6. The system of claim 1, further comprising a user interface coupled to the data processing system, wherein the data processing system is configured to receive user instructions for altering the gamma wave frequency signal via the user interface.

7. The system of claim 1, wherein integrating the altered gamma wave frequency signal into a musical composition comprises at least one of modulating an amplitude or a frequency of the altered gamma wave frequency signal.

8. The system of claim 1, wherein integrating the altered gamma wave frequency signal into a musical composition comprises layering the altered gamma wave frequency signal into the musical composition as a background
